(19) United States
(12) Patent Application Publication
(10) Pub. No.: US 2014/0228830 A1
(43) Pub. Date: Aug. 14, 2014

(54) Apparatus And Method For Sealing Of Tissue In Uncontrollable Bleeding Situations During Surgery

(71) Applicant: Jay Eunjae Kim, Issaquah, WA (US)

(72) Inventor: Jay Eunjae Kim, Issaquah, WA (US)

(21) Appl. No.: 14/070,568

(22) Filed: Nov. 3, 2013

Related U.S. Application Data
(60) Provisional application No. 61/763,920, filed on Feb. 12, 2013.
(60) Provisional application No. 61/772,514, filed on Mar. 4, 2013.

(51) Int. Cl. A61B 18/20

(52) U.S. Cl. CPC A61B 18/20
USPC 606/11; 606/10

(57) ABSTRACT

Embodiments of an apparatus and method for sealing of tissue during uncontrollable bleeding situations during surgery are described. In one aspect, an apparatus comprises a laser unit and a laser beam delivery unit. The laser unit is configured to emit a laser beam suitable for sealing an opening on a tissue. The laser beam delivery unit is detachably coupled to the laser unit, and is configured to guide and direct the laser beam to seal the opening on the tissue.

### CROSS REFERENCE TO RELATED PATENT APPLICATIONS

This is a non-provisional application claiming the priority benefit of two provisional applications, U.S. Patent Application No. 61/763,920 filed on Feb. 12, 2013 and U.S. Patent Application No. 61/772,514 filed on Mar. 4, 2013, which are incorporated by reference in their entirety.

### BACKGROUND

1. Technical Field

The present disclosure generally relates to the field of medical device and, more particularly, to a tissue sealer device using laser to seal tissue in uncontrollable bleeding situations during surgery.

2. Description of the Related Art

Presently, uncontrollable bleeding during surgery is addressed with electrocauterization devices such as monopolar and bipolar systems based on high electric currents. However, there is an intrinsic risk in the use of electrocauterization devices in an operating room due to exposure of high electric currents to patients and medical staff. As an electric current traverses through the body of a patient when an electrocauterization system is used, whether monopolar or bipolar, electric shock marks or burn marks on the patient (e.g., on the ear) tend to result. For example, monopolar systems can cause thermal injury to surrounding tissues and accidental burns if used incorrectly. One main issue with monopolar devices is the interference with automatic implantable cardioverter defibrillator (AICD) as the monopolar device may trigger accidental shock to the patient. To avoid accidental triggering, the AICD may need to be turned off prior to the use of a monopolar device on the patient. Additionally, the AICD may need to be re-programmed afterwards.

Moreover, the use of saline solution during surgery tends to be limited when a high electric current system is in use. Further, the use of electrocauterization systems may not be suitable for patients with metal implants.

### SUMMARY

Various embodiments disclosed herein pertain to an apparatus and method for sealing of tissue during uncontrollable bleeding situations during surgery. The apparatus comprises a laser unit and a laser beam delivery unit. The laser unit emits tunable laser beam with a wavelength suitable for sealing of a soft tissue. The laser beam delivery unit is disposable and is used to guide the laser beam to where tissue sealing is needed. Areas surrounding the tissue where tissue sealing occurs are cooled by a liquid dispensed from a probe of the laser beam delivery unit. Interference on the laser beam by the dispensed liquid is avoided or minimized by air flow provided to the tip of the probe. When energy level of reflected energy of the laser beam reaches a predefined level, the laser unit may deactivate the emission of the laser beam. At least one embedded sensor controls an on/off switch of the laser unit to ensure safety. Besides elimination of electric shock hazard, another benefit of the apparatus is that patients with metal implants and/or a pacemaker would have a safer option in surgery.

According to one aspect, an apparatus may comprise a laser unit and a laser beam delivery unit. The laser unit may be configured to emit a laser beam suitable for sealing an opening on a tissue. The laser beam delivery unit may be detachably coupled to the laser unit, and may be configured to guide and direct the laser beam to seal the opening on the tissue.

In one embodiment, the laser unit may be configured to emit the laser beam at a consistent energy level.

In one embodiment, at least one parameter of the laser beam may be tunable.

In one embodiment, the at least one parameter of the laser beam that is tunable may comprise a frequency of the laser beam, a power level of the laser beam, or a combination thereof.

In one embodiment, the laser beam may be tunable to be suitable for cutting the tissue open.

In one embodiment, the laser unit may comprise a laser source comprising a solid state laser or a fiber laser.

In one embodiment, the laser unit may emit the laser beam in a pulsed mode.

In one embodiment, the laser beam delivery unit may comprise a fiber optic tube and a probe. The fiber optic tube may be coupled to the laser unit, and may be configured to function as a guide for the laser beam. The probe may be coupled to the fiber optic tube, and may be configured to direct the laser beam toward a direction in which the probe is pointed.

In one embodiment, the laser beam delivery unit may further comprise a control unit coupled between the fiber optic tube and the probe. The control unit may include a switch that is configured to turn on the laser unit when in a first position and to turn off the laser unit when in a second position.

In one embodiment, the probe may comprise a connection end and a distal end. The connection end may be coupled to the fiber optic tube. The distal end may comprise a laser beam emitting port and at least one liquid dispensing port.

In one embodiment, the at least one liquid dispensing port of the probe may comprise a plurality of liquid dispensing ports surrounding the laser beam emitting port.

In one embodiment, the apparatus may further comprise a liquid pumping unit coupled to the laser beam delivery unit and a source of a liquid. The liquid pumping unit may be configured to pump the liquid to the laser beam delivery unit. The laser beam delivery unit may be configured to dispense the liquid through the at least one liquid dispensing port.

In one embodiment, the liquid may comprise saline or water.

In one embodiment, the liquid pumping unit may comprise a peristaltic pump.

In one embodiment, the apparatus may further comprise a compressed air unit coupled to the laser beam delivery unit. The compressed air unit may be configured to provide compressed air to the laser beam delivery unit. The laser beam delivery unit may be configured to discharge the compressed air through the distal end of the probe.

In one embodiment, the compressed air unit may comprise a compressed air reservoir or a compressed air pump.

In one embodiment, the apparatus may further comprise at least one sensor coupled to the probe of the laser beam delivery unit. The at least one sensor may be configured to sense a parameter associated with the tissue or the probe.

In one embodiment, the at least one sensor may be configured to approximately sense a temperature of the tissue or a surrounding thereof, a tilt angle of the probe, a distance between the distal end of the probe and the tissue, an optical characteristic associated with the tissue, or a combination thereof.

In one embodiment, the laser unit may be turned on and off at least in part based on an output of the at least one sensor.

In one embodiment, the at least one sensor may provide at least one indication indicative of a status of the tissue or the probe.

In one embodiment, the apparatus may further comprise a detector configured to detect at least a part of a reflection of the laser beam.

In one embodiment, the detector may comprise a photodiode.

According to another aspect, a method may comprise aiming a laser beam at an opening of a tissue, and applying a photosensitive material to an area of the tissue where the opening is located. The photosensitive material may be configured to aid sealing of the opening of the tissue.

In one embodiment, at least one parameter of the laser beam may be tunable. The at least one parameter of the laser beam that is tunable may comprise a frequency of the laser beam, a power level of the laser beam, or a combination thereof. The laser beam may also be tunable to be suitable for cutting the tissue open.

In one embodiment, the photosensitive material may comprise an adhesive material that enhances seal integrity of the tissue and promotes seal acceleration.

According to a further aspect, a method may provide a flow of a liquid to an area around an opening on a tissue. The method may also provide a flow of compressed air to the area around the opening on the tissue. The method may further activate emission of a laser beam at the opening on the tissue. The flow of the liquid may cool the area around the opening on the tissue. The flow of the compressed air may minimize interference of the laser beam by the flow of liquid by creating a dry chamber region above the opening on the tissue. The laser beam may be suitable for sealing the opening on the tissue.

In one embodiment, the liquid may comprise saline or water.

In one embodiment, the method may further measure an energy level of reflected energy of the laser beam. In response to the energy level of the reflected energy of the laser beam reaching a predefined level, the method may deactivate the emission of the laser beam.

The proposed techniques are further described below in the detailed description. This summary is not intended to identify essential features of the claimed subject matter, nor is it intended for use in determining the scope of the claimed subject matter.

### BRIEF DESCRIPTION OF THE DRAWINGS

The accompanying drawings are included to provide a further understanding of the disclosure, and are incorporated in and constitute a part of the present disclosure. The drawings illustrate embodiments of the disclosure and, together with the description, serve to explain the principles of the disclosure. It is appreciable that the drawings are not necessarily in scale as some components may be shown to be out of proportion than the size in actual implementation in order to clearly illustrate the concept of the present disclosure.

FIG. 1 is a tissue sealer apparatus in accordance with an embodiment of the present disclosure.

FIG. 2 is an enlarged partial view of the tissue sealer apparatus of FIG. 1 emitting a laser beam at a tissue.

FIG. 3 is an enlarged view of a probe tip of the tissue sealer apparatus of FIG. 1.

FIG. 4 shows air flow and liquid cooling at the probe tip of the tissue sealer apparatus of FIG. 1.

FIG. 5 shows optical feedback at the probe tip of the tissue sealer apparatus of FIG. 1.

FIG. 6 is a flowchart of a process of sealing a tissue with an apparatus in accordance with the present disclosure.

FIG. 7 is a flowchart of another process of sealing a tissue with an apparatus in accordance with the present disclosure.

### DETAILED DESCRIPTION OF PREFERRED EMBODIMENTS

### Overview

Various embodiments disclosed herein pertain to an apparatus and method for sealing of tissue during uncontrollable bleeding situations during surgery. The apparatus comprises a laser unit and a laser beam delivery unit. The laser unit emits tunable laser beam with a wavelength suitable for sealing of a soft tissue. The laser beam delivery unit is disposable and is used to guide the laser beam to where tissue sealing is needed. Areas surrounding the tissue where tissue sealing occurs are cooled by a liquid dispensed from a probe of the laser beam delivery unit. Interference on the laser beam by the dispensed liquid is avoided or minimized by air flow provided to the tip of the probe. When energy level of reflected energy of the laser beam reaches a predefined level, the laser unit may deactivate the emission of the laser beam. At least one embedded sensor controls an on/off switch of the laser unit to ensure safety. Besides elimination of electric shock hazard, another benefit of the apparatus is that patients with metal implants and/or a pacemaker would have a safer option in surgery.

Therefore, an apparatus in accordance with the present disclosure provides a number of advantages over electrocauterization devices and other existing approaches. The advantageous include, but are not limited to, the following:

(1) consistent power delivery;

(2) liquid cooling of tissues surrounding the opening;

(3) elimination of tissue charring and sticking;

(4) monitoring of tissue condition;

(5) intuitive touch and sealing technology;

(6) multiple sensors to ensure laser safety;

(7) manual and automatic power delivery; and

(8) various laser power sealing modes.

### Illustrative Implementations

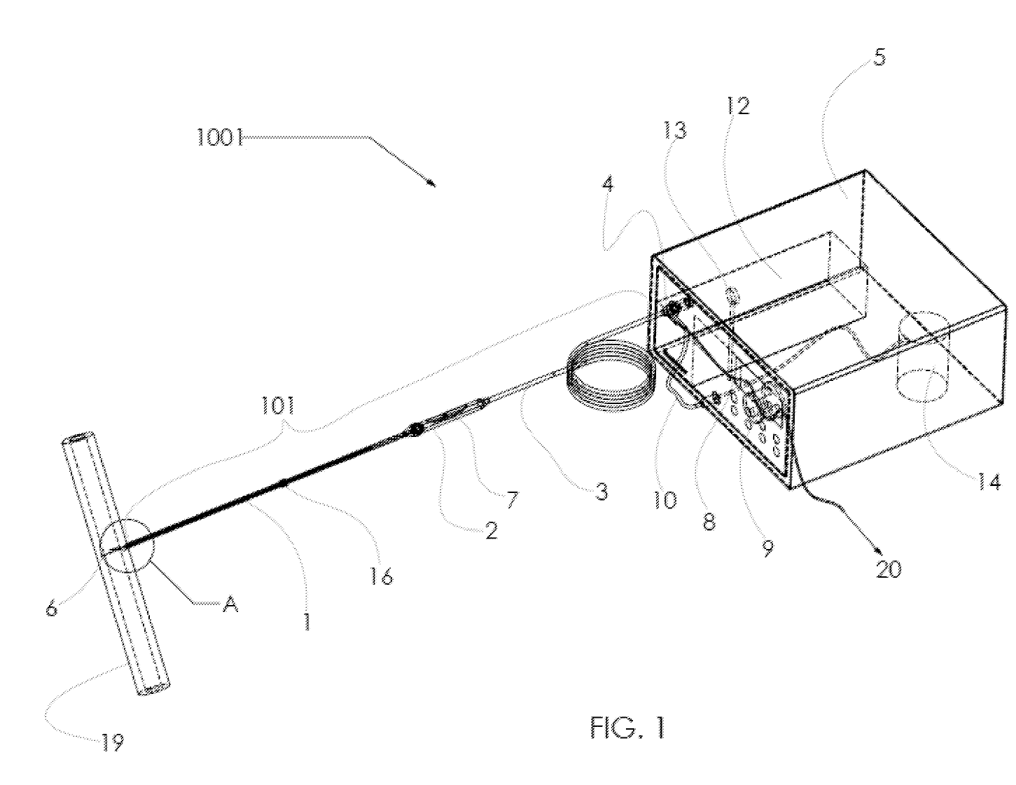

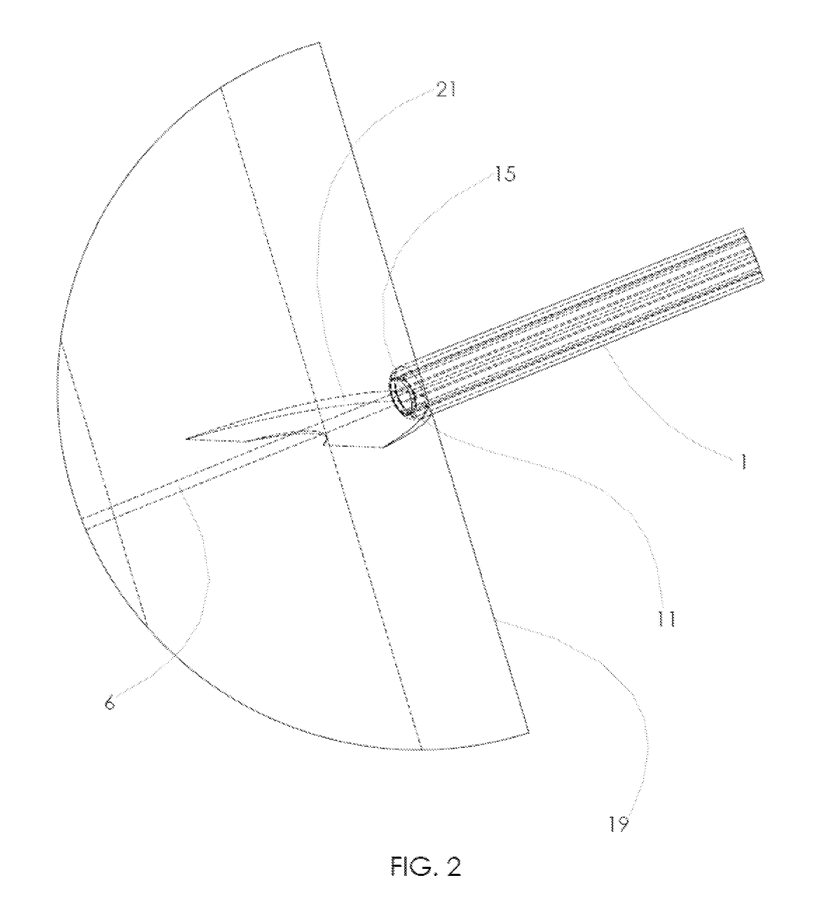

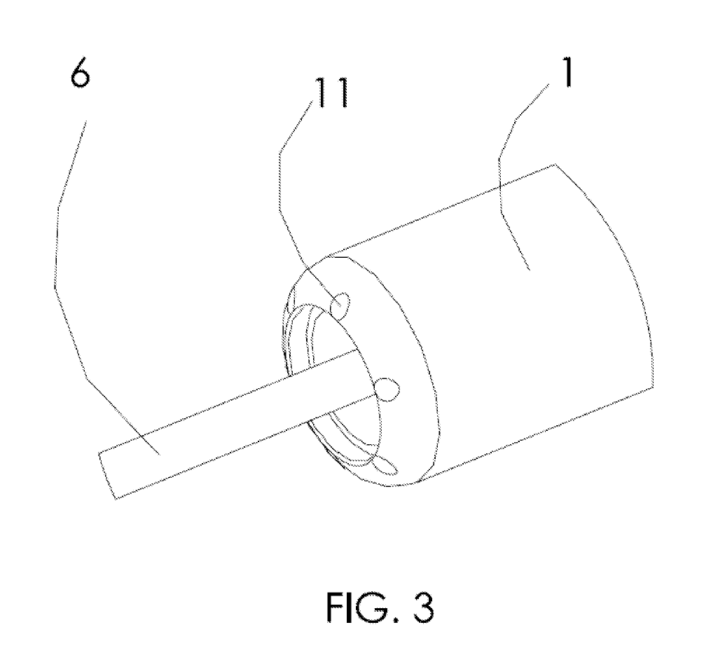

FIG. 1 illustrates a tissue sealer apparatus 1001 in accordance with an embodiment of the present disclosure. FIG. 2 is an enlarged partial view of the tissue sealer apparatus 1001 emitting a laser beam at a tissue. FIG. 3 is an enlarged view of a probe tip of the tissue sealer apparatus 1001. The following description refers to FIGS. 1-3.

As shown in FIG. 1, apparatus 1001 may comprise a laser unit 5 and a laser beam delivery unit 101. The laser unit 5 may comprise a laser source 12 that is configured to emit a laser beam 6 suitable for sealing an opening 21 on a tissue 19. The laser unit 5 is configured to emit the laser beam 6 at a consistent power or energy level. The laser beam delivery unit 101 may be detachably coupled to the laser unit 5, e.g., at a laser coupling port 4 on the laser unit 5. The laser beam delivery unit 101 may be configured to guide and direct the laser beam 6 to seal the opening 21 on the tissue 19. The laser beam delivery unit 101 may be disposable such that after each use it may be disposed of while the laser unit 5 is made to be durable and re-usable.

In one embodiment, at least one parameter of the laser beam 6 may be tunable. For example, a frequency of the laser beam 6, a power level of the laser beam 6, or a combination thereof, may be tunable.

In one embodiment, the laser beam 6 may be tunable to be suitable for cutting the tissue 19 open. For example, the laser unit 5 may be configured such that the laser beam 6 is tunable to a suitable wavelength and/or power level and/or pulsing frequency for the laser beam to cut the tissue 19 open and seal an opening 21 on the tissue 19. In other words, apparatus 1001 may be dual-functional in that it is not only configured to seal an open wound on the tissue 19 but also cut the tissue 19 open for surgical purposes.

In one embodiment, the laser source 12 may comprise a solid state laser, a fiber laser, or a combination thereof. In other embodiments, the laser unit 5 may comprise a gas laser, a photonic crystal laser, a semiconductor laser, a dye laser, a free electron laser, a bio laser, or a combination thereof.

In one embodiment, the laser unit 5 may emit the laser beam 6 in a pulsed mode.

In one embodiment, the laser beam delivery unit 101 may comprise a fiber optic tube 3 and a probe 1. The fiber optic tube 3 may be coupled to the laser unit 5, e.g., at the laser coupling port 4 on the laser unit 5, and may be configured to function as a guide for the laser beam 6. The probe 1 may be coupled to and surrounding at least a part of the fiber optic tube 3, and may be configured to direct the laser beam 6 toward a direction in which the probe 1 is pointed.

In one embodiment, the laser beam delivery unit 101 may further comprise a control unit 2 coupled between the fiber optic tube 3 and the probe 1. The control unit 2 may have a switch 7 such that the control unit 2 turns on the laser unit 5 when the switch 7 is in a first position (e.g., ON position) and turns off the laser unit 5 when the switch 7 is in a second position (e.g., OFF position).

In one embodiment, the probe 1 may comprise a connection end and a distal end. The connection end may be coupled to the fiber optic tube 3 or the control unit 2. The distal end may comprise a laser beam emitting port 15 from which the laser beam 6 is emitted.

In one embodiment, the apparatus 1001 may further comprise at least one sensor coupled to the probe 1 of the laser beam delivery unit 101. The at least one sensor may be configured to sense a parameter associated with the tissue 19 or the probe 1.

In one embodiment, the at least one sensor may be configured to approximately sense a temperature of the tissue 19 or a surrounding thereof, a tilt angle of the probe 1, a distance between the distal end of the probe 1 and the tissue 19, an optical characteristic associated with the tissue 19, or a combination thereof.

In one embodiment, the laser unit 5 may be turned on and off at least in part based on an output of the at least one sensor.

In one embodiment, the at least one sensor may provide at least one indication indicative of a status of the tissue 19 and/or the probe 1. In other words, the at least one sensor can monitor the condition of the tissue 19 and/or the probe 1. For example, the apparatus 1001 may further comprise a user output unit that displays an indication of one or more parameters sensed by the at least one sensor. In the example shown in FIG. 1, the probe 1 includes a light indicator 16 that can emit light or one or more colors to respectively provide one or more indications to the user. The light indicator 16 may comprise one or more light-emitting diodes (LEDs). Alternatively, the user output unit may play one or more sounds corresponding to one or more parameters sensed by the at least one sensor. In one embodiment, the user output unit may be a part of the laser unit 5.

In one embodiment, the probe 1 of the laser beam delivery unit 101 of apparatus 1001 may further comprise at least one liquid dispensing port 11. In one embodiment, the at least one liquid dispensing port 11 of probe 1 may comprise a plurality of liquid dispensing ports surrounding the laser beam emitting port.

In one embodiment, the apparatus 1001 may further comprise a liquid pumping unit 9 coupled to the laser beam delivery unit 101 and a source of a liquid, e.g., via a liquid tubing 20. The apparatus 1001 may further comprise tubing 8 coupled between the liquid pumping unit 9 and the laser beam delivery unit 101. The liquid pumping unit 9 may be configured to pump the liquid to the laser beam delivery unit 101 through tubing 8. The laser beam delivery unit 101 may be configured to dispense the liquid through the at least one liquid dispensing port 11.

In one embodiment, the liquid may comprise saline or water.

In one embodiment, the liquid pumping unit 9 may comprise a peristaltic pump.

In one embodiment, the apparatus 1001 may further comprise a compressed air unit 14 coupled to the laser beam delivery unit 101, e.g., via an air tubing 10. The compressed air unit 14 may be configured to provide compressed air to the laser beam delivery unit 101, and the laser beam delivery unit 101 may be configured to blow or otherwise discharge the compressed air out of the distal end of the probe 1.

In one embodiment, the compressed air unit 14 may comprise a compressed air reservoir or a compressed air pump.

In one embodiment, the apparatus 1001 may further comprise a detector 13 configured to detect at least a part of a reflection of the laser beam 6.

In one embodiment, the detector 13 may comprise a photodiode.

### Illustrative Operations

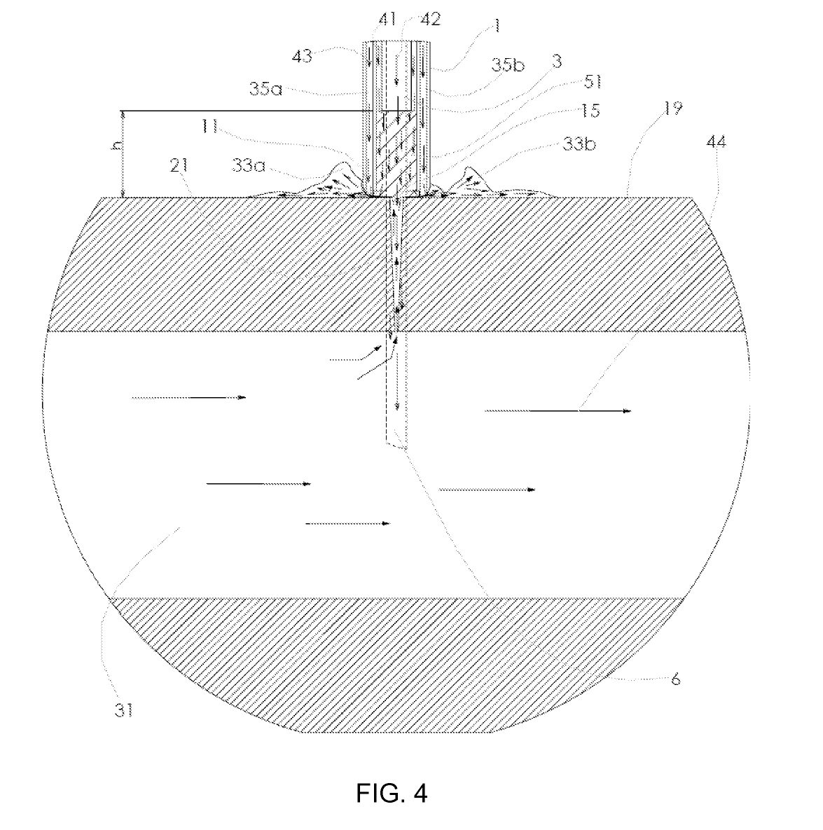

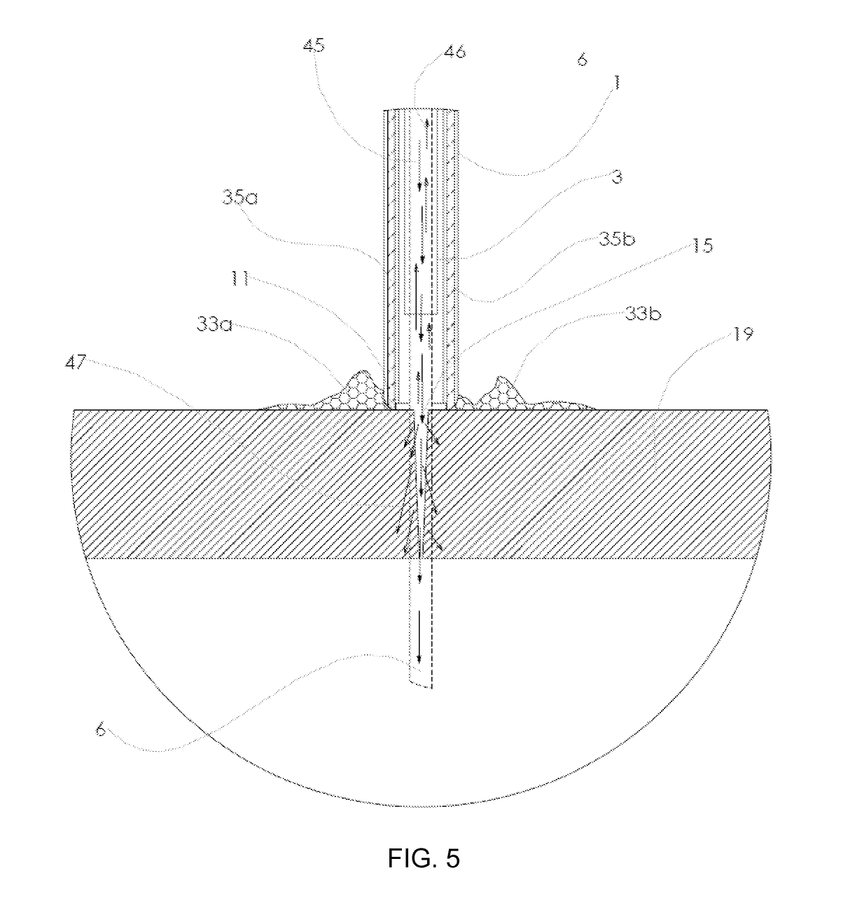

FIG. 4 shows air flow and liquid cooling at the probe tip of the tissue sealer apparatus 1001. FIG. 5 shows optical feedback at the probe tip of the tissue sealer apparatus 1001. The description below pertains to FIGS. 4 and 5.

As shown in FIG. 4, arrows 42 indicate the direction of the laser beam 6, arrows 41 indicate the direction of air flow of the compressed air, arrows 43 indicate the direction of liquid flow of the liquid (e.g., saline or water), and arrows 44 indicate the direction of blood flow of blood of the tissue 19.

In operation, the user may hold the laser beam emitting port 15 at the distal end of the probe 1 close to, almost touching or touching the tissue 19. The compressed air flows through a gap or spacing between an inner surface of the probe 1 and an outer surface of the fiber optic tube 3. In addition, the liquid (e.g., saline or water) flows through internal channels (e.g., channels 35a and 35b shown in FIG. 4) inside the wall of the probe 1. A mixture 33a, 33b of the blood, liquid and air is formed and may flow along an outer surface of the tissue 19 and away from the opening 21.

As shown in FIG. 4, when the laser beam emitting port 15 at the distal end of the probe 1 is held approximately touching but not completely touching the tissue 19 (e.g., with the laser beam emitting port 15 partially blocked by the tissue 19), a distal end of the fiber optic tube 3 is recessed approximately a distance h away from the tissue 19. Accordingly, there is a dry chamber region 51 inside and near the distal end of the probe 1 as there is little or no liquid in the dry chamber region 51 since air flow of the compressed air keeps the liquid (e.g., saline or water) away from the dry chamber region 51. With little or no liquid in the dry chamber region 51 due to the air flow, any interference on the laser beam 6 by the liquid (e.g., reflection, refraction, diffraction, etc.) can thus be avoided or otherwise minimized. In one embodiment, air flow of the compressed air is provided first to keep the dry camber region 51 dry or at least somewhat dry before the laser beam 6 is emitted. Meanwhile, the flow of liquid (e.g., saline or water) keeps areas of the tissue 19 surrounding the opening 21 cool by convection cooling to remove heat to avoid or otherwise minimize burning or thermal injuries to those areas of the tissue 19 surrounding the opening 21.

The apparatus 1001 is configured to discharge, dispense and emit the air flow, liquid flow and laser beam 6 separately or in combination. For example, all three of the air flow, liquid flow and laser beam 6 may be discharged, dispensed and emitted at a given time. Alternatively, only two of the air flow, liquid flow and laser beam 6 may be discharged, dispensed or emitted at a given time. In some cases, only one of the air flow, liquid flow and laser beam 6 may be discharged, dispensed or emitted at a given time.

In one embodiment, both the air flow and liquid flow are provided during emission of the laser beam 6. In another embodiment, none of the air flow and liquid flow is provided when the laser beam 6 is emitted. In yet another embodiment, either of the air flow and liquid flow is provided when the laser beam 6 is emitted.

The power or energy level of the laser beam 6 may be adjusted according to the tissue to be sealed. This may be done manually or automatically. For example, the user may manually adjust the energy level of the laser beam 6 by slowly or incrementally increasing the energy level from zero to a suitable level. Alternatively, the laser unit 5 may slowly or incrementally increase the energy level of the laser beam 6 from zero and monitors energy level of the reflected energy of the laser beam 6 as reflected from the tissue 19. The laser unit 5 may set the energy level of the laser beam 6 at a particular level or deactivate emission of the laser beam 6 when the energy level of the reflected energy of the laser beam 6 reaches a predefined level.

The liquid pumping unit 9, the compressed air unit 14 and the laser source 12 may be activated/deactivated (e.g., turned on/off) separately or in combination at the switch 7 or at one or more other switches on the apparatus 1001.

As shown in FIG. 5, arrows 45 indicate the direction of the full laser beam 6, arrows 46 indicate the direction of reflected energy of the laser beam 6, and arrows 47 indicate the directions of absorbed laser beam after the laser beam 6 reaches the tissue 19.

The reflected energy of the laser beam 6 (indicated by the arrows 46) is detected by the detector 13. Energy level of the reflected energy of the laser beam 6 is measured (e.g., by a processor in the laser unit 5) to determine presence of blood or liquid and/or seal quality of the opening 21 by comparing energy level versus tissue texture of the tissue 19. In one embodiment, optical signal may be detected at a frequency less than 1 Hz.

A feedback signal may be provided to the user (e.g., a surgeon) in terms of color light signal, e.g., by the light indicator 16 which is attached on probe 1. The apparatus 1001 can alert the user that sealing of the tissue 19 is completed or that bleeding has been stopped so that the user can move the probe 1 to another bleeding area (whether or not on the tissue 19) to perform additional sealing operations.

The apparatus 1001 may be operated in a manual mode and in an automatic mode. To illustrate, consider a case in which a surgeon operates the apparatus 1001 in the manual mode. The surgeon may push the switch 7 to turn on/off while holding the switch 7. The surgeon may bring the laser beam emitting port 15 of the probe 1 close to the bleeding tissue 19 of a patient, e.g., to approximately ¼ inch or less away, and turn on both air flow and liquid flow. When the surgeon touches the tissue 19 with the tip or distal end of the probe 1, the surgeon may activate the laser source 12 of the laser unit 5 to emit the laser beam 6 to seal the opening 21 on the tissue 19. After the surgeon verifies that there is no more bleeding, the surgeon may move to the next bleeding area of the patient. The manual mode of operation may be suitable for experienced surgeons.

For less-experienced surgeons, the apparatus 1001 may be operated in the automatic mode. More specifically, when the surgeon moves the tip or distal end of the probe 1 to touch the tissue 19, the laser source 12 may be automatically activated to emit the laser beam 6. The detector 13 in the laser unit 5 monitors reflected energy of the laser beam 6 for a processor in the laser unit 5 to determine whether sealing of the opening 21 of the tissue 19 is completed. When it is determined that the sealing of the opening 21 is completed, the laser unit 5 may indicate to the surgeon, e.g., via one or more light signals and/or one or more audio signals, to let the surgeon know that the sealing of the opening 21 is completed.

### Illustrative Processes

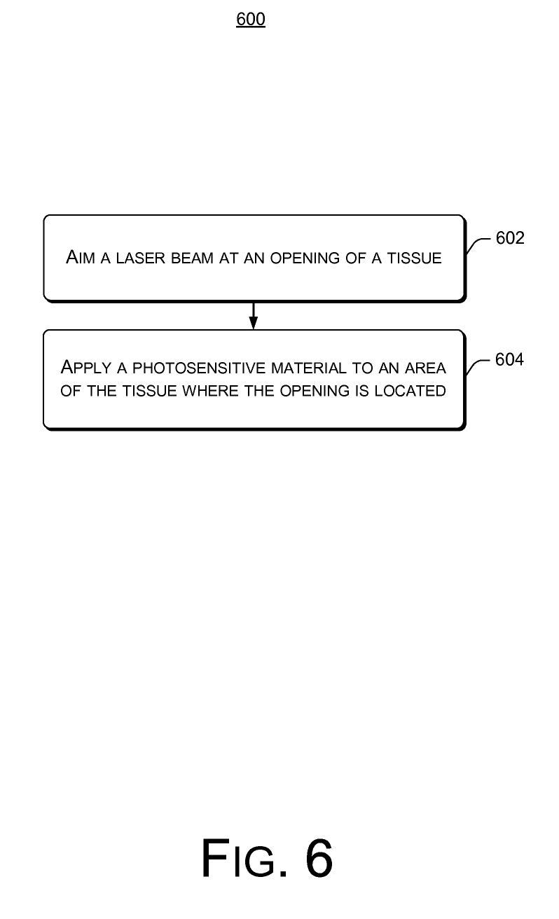

FIG. 6 illustrates a process 600 of sealing a tissue with an apparatus in accordance with the present disclosure.

Example process 600 includes one or more operations, actions, or functions as illustrated by one or more of blocks 602 and 604. Although illustrated as discrete blocks, various blocks may be divided into additional blocks, combined into fewer blocks, or eliminated, depending on the desired implementation. Further, process 600 may be implemented using apparatus 1001. For illustrative purposes, the operations described below are performed by medical personnel using apparatus 1001. Process 600 may begin at block 602.

At 602, process 600 may comprise aiming a laser beam at an opening of a tissue. For example, the laser beam 6 may be aimed at the tissue 19 to seal the opening 21.

At 604, process 600 may comprise applying a photosensitive material to an area of the tissue where the opening is located. The photosensitive material may be configured to aid sealing of the opening of the tissue. The photosensitive material may be applied during and/or after the laser beam is aimed at the opening of the tissue.

In one embodiment, at least one parameter of the laser beam may be tunable. The at least one parameter of the laser beam that is tunable may comprise a frequency of the laser beam, a power level of the laser beam, or a combination thereof. The laser beam may also be tunable to be suitable for cutting the tissue open.

In one embodiment, the photosensitive material may comprise an adhesive material that enhances seal integrity of the tissue and promotes seal acceleration.

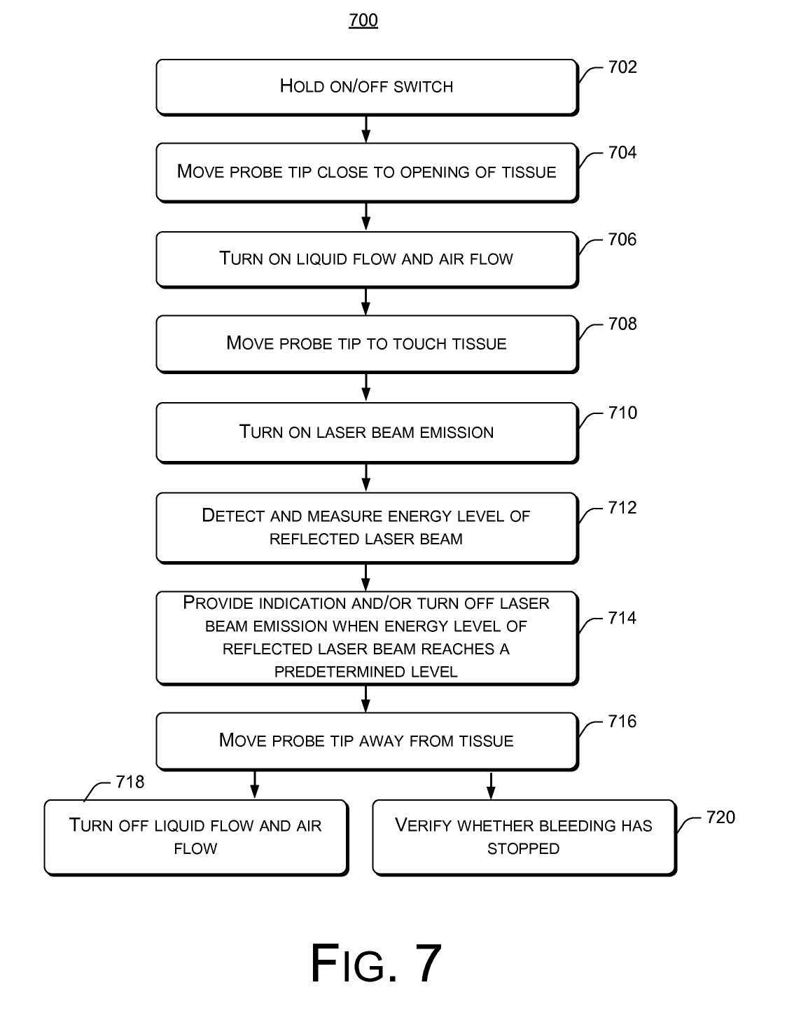

FIG. 7 illustrates a process 700 of sealing a tissue with an apparatus in accordance with the present disclosure.

Example process 700 includes one or more operations, actions, or functions as illustrated by one or more of blocks 702-720. Although illustrated as discrete blocks, various blocks may be divided into additional blocks, combined into fewer blocks, or eliminated, depending on the desired implementation. Further, process 700 may be implemented using apparatus 1001. For illustrative purposes, the operations described below are performed by medical personnel, e.g., a surgeon, using apparatus 1001. Process 700 may begin at block 702.

At 702, the surgeon holds the on/off switch 7 on probe 1 of the apparatus 1001.

At 704, the surgeon moves the tip or distal end of probe 1 of the apparatus 1001 close to the opening 21 of the tissue 19, e.g., to a distance ¼ inch or less away from the tissue 19.

At 706, the surgeon turns on the liquid pumping unit 9 and the compressed air unit 14 to start dispending liquid and discharging air from the distal end of the probe 1.

At 708, the surgeon moves the tip of probe 1 to touch the tissue 19.

At 710, the surgeon turns on the laser source 12 to emit the laser beam 6 at the opening 21 of the tissue 19.

At 712, the detector 13 detects energy level of reflected energy of the laser beam 6 and a processor of the laser unit 5 measures the energy level to determine whether or not the measured energy level has reached a predefined level. As the opening 21 of the tissue 19 is sealed, more energy of the laser beam 6 is reflected back. Accordingly, it is deemed that sealing of the opening 21 is completed when the energy level of the reflected energy has reached the predefined level.

At 714, when it is determined that the measured energy level has reached the predefined level, the apparatus 1001 may provide a visual and/or audio signal to the surgeon to notify the surgeon that sealing of the opening 21 is completed. Additionally or alternatively, the laser source 12 is turned off to stop emission of the laser beam 6. This may be done manually by the surgeon using the switch 7 or automatically by laser unit 5.

At 716, the surgeon moves the tip of probe 1 away from the opening 21 of the tissue 19.

At 718, the surgeon turns off the liquid flow and air flow.

At 720, the surgeon verifies whether bleeding from the tissue 19 is stopped (i.e., the opening 21 has been sealed). If bleeding is not stopped, the surgeon may repeat the previous operations to stop the bleeding by completely sealing the opening 21.

From the perspective of the apparatus 1001, the apparatus 1001 may provide a flow of a liquid (e.g., saline or water) and a flow of compressed air to an area around the opening 21 on the tissue 19. The apparatus 1001 may also activate emission of the laser beam 6 at the opening 21 on the tissue 19. The flow of the liquid cools the area around the opening 21 on the tissue 19. The flow of the compressed air minimizes interference of the laser beam 6 by the flow of liquid by creating a dry chamber region above the opening 21 on the tissue 19. The laser beam 6 is at an appropriate frequency and energy level suitable for sealing the opening 21 on the tissue 19.

When operating in the automatic mode, the apparatus 1001 may measure an energy level of reflected energy of the laser beam 6. In response to the energy level of the reflected energy of the laser beam 6 reaching a predefined level, the apparatus 1001 may deactivate the emission of the laser beam 6.

### Additional and Alternative Implementation Notes

The above-described embodiments and techniques pertain to an apparatus and method for sealing of tissue during uncontrollable bleeding situations during surgery. Although the techniques have been described in language specific to certain applications, it is to be understood that the appended claims are not necessarily limited to the specific features or applications described herein. Rather, the specific features and applications are disclosed as example forms of implementing such techniques.

In the above description of example implementations, for purposes of explanation, specific numbers, materials configurations, and other details are set forth in order to better explain the invention, as claimed. However, it will be apparent to one skilled in the art that the claimed invention may be practiced using different details than the example ones described herein. In other instances, well-known features are omitted or simplified to clarify the description of the example implementations.

The described embodiments are intended to be primarily examples. The described embodiments are not meant to limit the scope of the appended claims. Rather, the claimed invention might also be embodied and implemented in other ways, in conjunction with other present or future technologies.

Moreover, the word “example” is used herein to mean serving as an example, instance, or illustration. Any aspect or design described herein as “example” is not necessarily to be construed as preferred or advantageous over other aspects or designs. Rather, use of the word example is intended to present concepts and techniques in a concrete fashion. The term “techniques,” for instance, may refer to one or more devices, apparatuses, systems, methods, articles of manufacture, and/or computer-readable instructions as indicated by the context described herein.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

1. An apparatus, comprising:
a laser unit configured to emit a laser beam suitable for sealing an opening on a tissue; and
a laser beam delivery unit detachably coupled to the laser unit, the laser beam delivery unit configured to guide and direct the laser beam to seal the opening on the tissue.

2. The apparatus of claim 1, wherein at least one parameter of the laser beam is tunable.

3. The apparatus of claim 2, wherein the at least one parameter of the laser beam that is tunable comprises a frequency of the laser beam, a power level of the laser beam, or a combination thereof.

4. The apparatus of claim 2, wherein the laser beam is tunable to be suitable for cutting the tissue open.

5. The apparatus of claim 1, wherein the laser unit comprises a laser source comprising a solid state laser or a fiber laser.

6. The apparatus of claim 1, wherein the laser unit emits the laser beam in a pulsed mode.

7. The apparatus of claim 1, wherein the laser beam delivery unit comprises:
a fiber optic tube coupled to the laser unit, the fiber optic tube configured to function as a guide for the laser beam; and
a probe coupled to and surrounding at least a portion of the fiber optic tube, the probe configured to direct the laser beam toward a direction in which the probe is pointed.

8. The apparatus of claim 7, wherein the laser beam delivery unit further comprises:
a control unit coupled between the fiber optic tube and the probe, the control unit having a switch that is configured to turn on the laser unit when in a first position and to turn off the laser unit when in a second position.

9. The apparatus of claim 7, wherein the probe comprises a connection end and a distal end, the connection end coupled to the fiber optic tube, the distal end comprising a laser beam emitting port and at least one liquid dispensing port.

10. The apparatus of claim 9, wherein the at least one liquid dispensing port comprises a plurality of liquid dispensing ports surrounding the laser beam emitting port.

11. The apparatus of claim 9, further comprising:
a liquid pumping unit coupled to the laser beam delivery unit and a source of a liquid, the liquid pumping unit configured to pump the liquid to the laser beam delivery unit, wherein the laser beam delivery unit is configured to dispense the liquid through the at least one liquid dispensing port.

12. The apparatus of claim 11, wherein the liquid comprises saline or water.

13. The apparatus of claim 11, wherein the liquid pumping unit comprises a peristaltic pump.

14. The apparatus of claim 9, further comprising:
a compressed air unit coupled to the laser beam delivery unit, the compressed air unit configured to provide compressed air to the laser beam delivery unit, wherein the laser beam delivery unit is configured to discharge the compressed air through the distal end of the probe.

15. The apparatus of claim 14, wherein the compressed air unit comprises a compressed air reservoir or a compressed air pump.

16. The apparatus of claim 7, further comprising:
at least one sensor coupled to the probe of the laser beam delivery unit, the at least one sensor configured to sense a parameter associated with the tissue or the probe.

17. The apparatus of claim 14, wherein the at least one sensor is configured to approximately sense a temperature of the tissue or a surrounding thereof, a tilt angle of the probe, a distance between the distal end of the probe and the tissue, an optical characteristic associated with the tissue, or a combination thereof.

18. The apparatus of claim 14, wherein the laser unit is turned on and off at least in part based on an output of the at least one sensor.

19. The apparatus of claim 14, wherein the at least one sensor provides at least one indication indicative of a status of the tissue or the probe.

20. The apparatus of claim 1, further comprising:
a detector configured to detect at least a part of a reflection of the laser beam.

21. (canceled)

22. (canceled)

23. (canceled)

24. (canceled)

25. (canceled)

26. (canceled)

27. (canceled)
